(19) United States
(12) Patent Application Publication
(10) Pub. No.: US 2025/0108227 A1
(43) Pub. Date: Apr. 3, 2025

(54) Transducer Arrays for Tumor Treating Fields (TTFields) Therapy That Use a Dedicated Flex Circuit to Implement Temperature Sensing

(71) Applicant: Novocure GmbH, Root D4 (CH)

(72) Inventors: Yoram WASSERMAN, Haifa (IL); Stas Obuchovsky, Haifa (IL); David Shapiro, Haifa (IL); Inbar Schlachet Drukerman, Haifa (IL)

(73) Assignee: Novocure GmbH, Root D4 (CH)

(21) Appl. No.: 18/899,353

(22) Filed: Sep. 27, 2024

Related U.S. Application Data
(60) Provisional application No. 63/541,419, filed on Sep. 29, 2023.

(51) Int. Cl. A61N 1/40

(52) U.S. Cl. CPC A61N 1/40

(57) ABSTRACT

Alternating electric fields (e.g., Tumor Treating Fields or TTFields) can be applied to a subject's body using an apparatus (e.g., a transducer array) that includes a first PCB that has metal pads for coupling an AC signal into the subject's body at high currents, and a second, less expensive PCB that can only handle lower currents. The first PCB is located at a central portion of the apparatus, and the second PCB is located at a peripheral portion of the apparatus. Temperature sensors (e.g., thermistors) are electrically connected to the second PCB. In some embodiments, the second PCB is significantly larger than the first PCB.

### CROSS REFERENCE TO RELATED APPLICATIONS

This application claims the benefit of U.S. Provisional Application 63/541,419, filed Sep. 29, 2023, which is incorporated herein by reference in its entirety.

### BACKGROUND

Tumor Treating Fields (TTFields) therapy is a proven approach for treating tumors using alternating electric fields at frequencies e.g., between 50 KHz-5 MHz, more commonly 100-500 KHz. Conventionally, the alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body. And higher currents are strongly correlated with higher efficacy of treatment.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier (BBB) so that, e.g., chemotherapy drugs can reach the brain.

One example of prior art electrode assemblies that can be used to apply alternating electric fields to a subject's body are the electrode assemblies described in U.S. Pat. No. 8,715,203. These electrode assemblies include nine electrode elements, each of which includes (a) a metal layer and (b) a ceramic layer with a very high dielectric constant positioned between the metal layer and the subject's skin. Thermistors positioned in small holes in the center of most of the electrode elements are included to make temperature measurements at those electrode elements.

Another example of prior art electrode assemblies that can be used to apply alternating electric fields to a subject's body are the electrode assemblies described in Pub. No. US 2021/0402179. These electrode assemblies have a flex circuit that includes a plurality of conductive pads on the front side of the flex circuit and a plurality of flexible polymer regions disposed over and in front of the conductive pads. A plurality of thermistors positioned on a rear side of the flex circuit in thermal contact with respective conductive pads are used to sense the temperature of the conductive pads.

To use either of these prior art electrode assemblies, an AC voltage is applied to the metal layers of the electrode elements in opposing electrode assemblies to generate the TTFields in the subject's body. The skin beneath the electrode elements heats up during use, resulting in a set of hotter spots immediately below the electrode elements, and a set of cooler regions immediately below the spaces between the electrode elements. And because safety considerations require that the skin temperature remains below a safety threshold (e.g., 41° C.), these hot spots limit the amount of current that can be delivered through the prior art electrode assemblies. More specifically, the prior art systems rely on signals from the thermistors to ensure that the current is low enough to prevent the temperature of the subject's skin from exceeding the safety threshold.

### SUMMARY OF THE INVENTION

One aspect of the invention is directed to a first apparatus for applying an electrical signal to a subject's body. The first apparatus comprises a first PCB, a second PCB, and a plurality of temperature sensors. The first PCB has a first substrate and at least one metal pad disposed on a skin-facing front face of the first substrate. The second PCB has a second substrate and a plurality of conductive traces, and the second PCB is flexible. Each of the plurality of temperature sensors is electrically connected to at least one of the conductive traces of the second PCB. The first PCB has an outer boundary, and the first and second PCBs are positioned so that, when viewed from a direction perpendicular to the first PCB, the second PCB lies outside the outer boundary of the first PCB.

Some embodiments of the first apparatus further comprise a flexible backing positioned behind the first PCB and the second PCB. The flexible backing is configured to support the first PCB and the second PCB, and at least a portion of the flexible backing extends laterally beyond both the first PCB and the second PCB and is covered with a biocompatible adhesive that adheres to skin. The first PCB is flexible, and the plurality of conductive traces of the second PCB are formed using conductive ink. Optionally, in these embodiments, the first PCB has a first area, the second PCB has a second area, and the second area is larger than the first area.

Some embodiments of the first apparatus further comprise a flexible backing positioned behind the first PCB and the second PCB. The flexible backing is configured to support the first PCB and the second PCB, and at least a portion of the flexible backing extends laterally beyond both the first PCB and the second PCB and is covered with a biocompatible adhesive that adheres to skin. The first PCB is flexible, and the plurality of conductive traces of the second PCB are formed using conductive ink. These embodiments also further comprise a first layer of conductive adhesive or conductive gel disposed on and in front of both the first PCB and the second PCB; a layer of anisotropic material disposed on and in front of the first layer of conductive adhesive or conductive gel; and a second layer of conductive adhesive or conductive gel disposed on and in front of the layer of anisotropic material.

Optionally, in the embodiments described in the previous paragraph, the layer of anisotropic material comprises graphite. Optionally, in the embodiments described in the previous paragraph, the layer of anisotropic material comprises a layer of pyrolytic graphite, graphitized polymer film, or graphite foil made from compressed high purity exfoliated mineral graphite. Optionally, the embodiments described in the previous paragraph may further comprise at least one layer of dielectric material disposed in front of the at least one metal pad (optionally disposed on and in front of the at least one metal pad), and the at least one layer of dielectric material has a dielectric constant of at least 10. Optionally, in the embodiments described in the previous paragraph, the first PCB further comprises at least one layer of dielectric material disposed on the at least one metal pad and in front of the at least one metal pad, and the at least one layer of dielectric material has a dielectric constant of at least 10. Optionally, each of the metal pads in the latter embodiments can be copper.

Some embodiments of the first apparatus further comprise a connector affixed to the second PCB. And the plurality of temperature sensors, the plurality of conductive traces of the second PCB, and the connector are positioned and arranged so that temperature readings obtained using the plurality of temperature sensors can be accessed via the connector.

In some embodiments of the first apparatus, the first PCB has a first area, the second PCB has a second area, and the second area is at least twice as large as the first area.

In some embodiments of the first apparatus, the first and second substrates partially or completely overlap one another in an overlap region and are laminated together in the overlap region. In some embodiments of the first apparatus, the first and second substrates are the same substrate or the first and second substrates are coplanar.

In some embodiments of the first apparatus, the plurality of temperature sensors comprises at least two temperature sensors, or at least three temperature sensors, or at least four temperature sensors.

Another aspect of the invention is directed to a second apparatus for applying an electrical signal to a subject's body. The second apparatus comprises a flexible PCB, at least two temperature sensors, a first layer of conductive adhesive or conductive gel, and a flexible backing. The flexible PCB has a central section and a peripheral section located outside an outer boundary of the central section, and the PCB has at least one metal pad disposed on a skin-facing front face of the central section of the PCB, and a plurality of conductive traces. Each of the at least two temperature sensors is mounted to the peripheral section of the PCB and electrically connected to at least one of the conductive traces. The first layer of conductive adhesive or conductive gel is disposed on and in front of the PCB. And the flexible backing is positioned behind the PCB. The flexible backing is configured to support the PCB, and at least a portion of the flexible backing extends laterally beyond the PCB and is covered with a biocompatible adhesive that adheres to skin. In some embodiments, the second apparatus comprises at least three temperature sensors, or at least four temperature sensors.

In some embodiments of the second apparatus, the central section of the flexible PCB is bounded by a convex hull enclosing a perimeter of outermost metal pads of the at least one metal pad.

Some embodiments of the second apparatus further comprise a layer of anisotropic material disposed on and in front of the first layer of conductive adhesive or conductive gel, and the layer of anisotropic material has an area that is at least twice as large as an area of the central section; and a second layer of conductive adhesive or conductive gel disposed on and in front of the layer of anisotropic material.

Optionally, in the embodiments described in the previous paragraph, the layer of anisotropic material comprises graphite. Optionally, in the embodiments described in the previous paragraph, the layer of anisotropic material comprises a layer of pyrolytic graphite, graphitized polymer film, or graphite foil made from compressed high purity exfoliated mineral graphite.

Some embodiments of the second apparatus further comprise at least one layer of dielectric material disposed in front of the at least one metal pad. The at least one layer of dielectric material has a dielectric constant of at least 10.

In some embodiments of the second apparatus, the PCB further comprises at least one layer of dielectric material disposed on the at least one metal pad and in front of the at least one metal pad, and the at least one layer of dielectric material has a dielectric constant of at least 10. In some embodiments of the second apparatus, each conductive trace in the peripheral section connecting to the at least two temperature sensors of the plurality of conductive traces is formed using conductive ink. In some embodiments of the second apparatus, each of the metal pads is a copper pad. In some embodiments of the second apparatus, none of the metal pads are located in the peripheral section of the PCB.

Another aspect of the invention is directed to a third apparatus for applying an electrical signal to a subject's body. The third apparatus comprises a first PCB, a second PCB, a plurality of temperature sensors, and a flexible backing. The first PCB has a first substrate and at least one metal pad disposed on a skin-facing front face of the first substrate. The second PCB has a second substrate and a plurality of conductive traces formed using conductive ink, and the second PCB is flexible. Each of the plurality of temperature sensors is electrically connected to at least one of the conductive traces of the second PCB. The flexible backing is positioned behind the first PCB and the second PCB, the flexible backing is configured to support the first PCB and the second PCB, and at least a portion of the flexible backing extends laterally beyond both the first PCB and the second PCB and is covered with a biocompatible adhesive that adheres to skin. The first PCB has an outer boundary, the second PCB has an outer boundary, and the first and second PCBs are positioned so that a convex hull that most closely circumscribes the outer boundary of the second PCB lies outside the outer boundary of the first PCB.

Some embodiments of the third apparatus further comprise a first layer of conductive adhesive or conductive gel disposed on and in front of both the first PCB and the second PCB; a layer of anisotropic material disposed on and in front of the first layer of conductive adhesive or conductive gel; and a second layer of conductive adhesive or conductive gel disposed on and in front of the layer of anisotropic material.

Optionally, in the embodiments described in the previous paragraph, the layer of anisotropic material comprises graphite. Optionally, in the embodiments described in the previous paragraph, the layer of anisotropic material comprises a layer of pyrolytic graphite, graphitized polymer film, or graphite foil made from compressed high purity exfoliated mineral graphite. Optionally, in the embodiments described in the previous paragraph, the first PCB further comprises at least one layer of dielectric material disposed on the at least one metal pad and in front of the at least one metal pad, and the at least one layer of dielectric material has a dielectric constant of at least 10. In some embodiments of the third apparatus, the plurality of temperature sensors comprises at least two temperature sensors, or at least three temperature sensors, or at least four temperature sensors.

### BRIEF DESCRIPTION OF THE DRAWINGS

FIG. 1 is a plan view of an apparatus for applying alternating electric fields (e.g., TTFields) to a subject's body.

FIG. 2 is a section view of the apparatus of FIG. 1.

FIG. 3 depicts an example of how to use the apparatus of FIG. 1 to apply alternating electric fields (e.g., TTFields) to a subject's body.

FIG. 4 is a plan view of another apparatus for applying alternating electric fields (e.g., TTFields) to a subject's body.

FIG. 5 is a section view of the apparatus of FIG. 4.

FIG. 6 depicts another apparatus for applying alternating electric fields (e.g., TTFields) to a subject's body.

FIG. 7 is a section view that depicts yet another apparatus for applying alternating electric fields (e.g., TTFields) to a subject's body.

FIG. 8 depicts an apparatus for applying alternating electric fields (e.g., TTFields) to a subject's body using two PCBs, where the two PCBs overlap.

FIG. 9 depicts another apparatus for applying alternating electric fields (e.g., TTFields) to a subject's body using two PCBs, where the two PCBs overlap.

Various embodiments are described in detail below with reference to the accompanying drawings, wherein like reference numerals represent like elements.

### DESCRIPTION OF THE PREFERRED EMBODIMENTS

Notably, in the two prior art designs described above, the hottest spots are always located directly beneath one of the electrode elements. It is therefore reasonable for these prior art systems to rely on temperature readings obtained at the electrode elements. But with other designs for transducer arrays, obtaining temperature measurements only at the locations of the electrode elements may be insufficient to ensure that no portions of the transducer array heat up beyond the safety threshold. This can be particularly important in designs that use sheets of heat-conductive materials (e.g., graphite) to spread the heat out within any given transducer array. Examples of this type of transducer array are described in Pub. No. US 2023/0043071, which is incorporated herein by reference in its entirety.

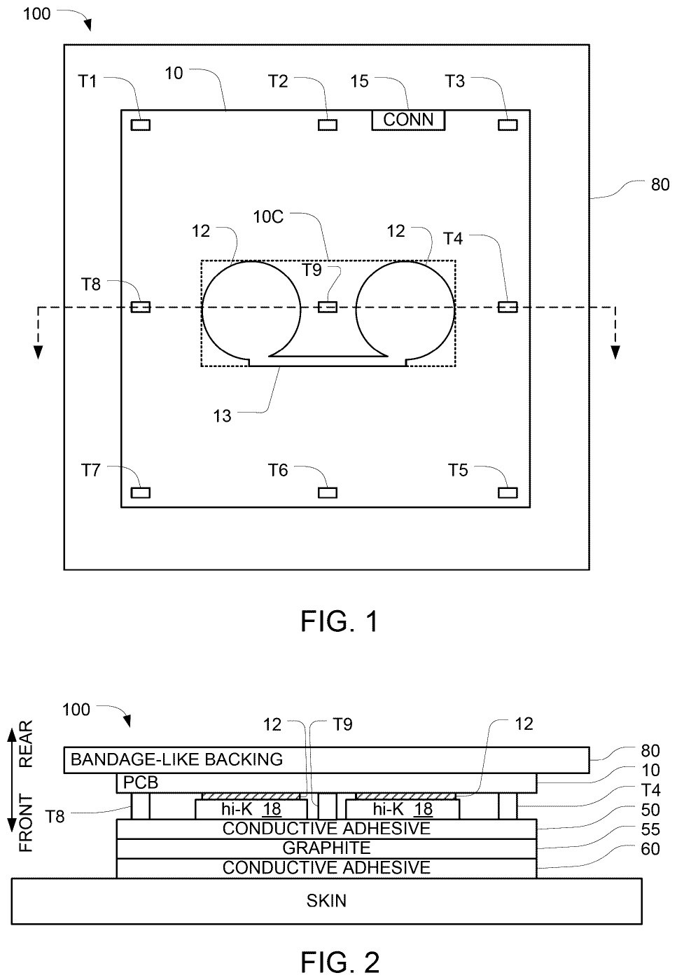

FIG. 1 is a plan view of an apparatus 100 (e.g., a transducer array) for applying alternating electric fields (e.g., TTFields) to a subject's body, and FIG. 2 is a section view of the same apparatus 100. The apparatus 100 includes at least one metal pad 12 positioned in the central portion of the apparatus, and a set of temperature sensors (e.g., thermistors) T1-T8 positioned at the peripheral portion of the apparatus is used to obtain temperature readings of the periphery of the apparatus.

The apparatus 100 uses a flexible PCB 10 that has a central section 10C and a peripheral section (i.e., the section that is located outside the outer boundary of the central section 10C). Note that as used herein, the term “PCB” refers to a printed circuit board, and this term encompasses rigid PCBs (e.g., with copper traces on a rigid epoxy board), flex circuits (e.g., with copper traces on a flexible polyimide substrate), and printed circuits made by printing a conductive ink on a flexible substrate.

The PCB 10 has at least one metal pad 12 disposed on a front face of the central section 10C of the PCB, and a plurality of conductive traces (not shown). At least two temperature sensors T1-T8 (e.g., thermistors) are mounted to the peripheral section of the PCB 10 (for example, four or more temperature sensors for electrode elements/metal pads distributed in a square or rectangular pattern), and these temperature sensors are electrically connected to at least one of the conductive traces. Optionally, one or more additional temperature sensors (e.g., thermistors) T9 may be positioned in the central portion of the apparatus 100. A connector 15 is mounted to the PCB 10, and this connector 15 is used to provide an electrical interface with the thermistors T1-T9 and the at least one metal pad 12. When more than one metal pad 12 is included (as depicted in FIGS. 1-2), all the metal pads 12 can be connected by conductive traces (e.g., metal traces) 13, in which case only a single pin of the connector 15 will be required to apply an AC signal to all of the metal pads 12. In alternative embodiments (not shown), the conductive traces 13 can be omitted, and each metal pad 12 can be electrically connected to its own individual pin on the connector 15.

In the embodiment depicted in FIGS. 1-2, the PCB 10 also includes a layer of dielectric material 18 disposed on and in front of the metal pads 12, and this layer of dielectric material 18 (labelled “hi-K” in FIG. 2) has a dielectric constant of at least 10. In some preferred embodiments, the layer of dielectric material 18 has a dielectric constant of at least 20 or at least 40. A first layer of conductive adhesive 50 can be disposed in front of the PCB 10. In FIGS. 1-2, the first layer of conductive adhesive 50 is shown disposed on and in front of the PCB 10, and the rear surface of this conductive adhesive 50 adheres to the front surface of the dielectric material 18 and also to the front surfaces of the thermistors T1-T8 (and T9, if present). Note that in some embodiments, the layer of dielectric material 18 is not included, in which case the rear surface of the first layer of conductive adhesive 50 will adhere to the front surface of the metal pads 12 and also to the front surfaces of the thermistors T1-T8 (and T9, if present). Note that in alternative embodiments, a layer of conductive gel (e.g., hydrogel) can be used in place of the first layer of conductive adhesive 50 depicted in FIG. 2.

A flexible backing 80 (e.g., a bandage-like backing) is positioned behind the PCB 10, and this flexible backing 80 is configured to support the PCB. At least a portion of the flexible backing 80 extends laterally beyond the PCB and the front of this portion is covered with a biocompatible adhesive that adheres to skin. This portion of the flexible backing 80 helps holds the apparatus 100 against the subject's skin.

The embodiment depicted in FIGS. 1-2 also includes a layer of anisotropic material (e.g., a sheet of graphite) 55 that is disposed on and in front of the first layer of conductive adhesive 50, and a second layer of conductive adhesive 60 disposed on and in front of the layer of anisotropic material 55. The layer of anisotropic material 55 can be a layer of pyrolytic graphite, graphitized polymer film, or graphite foil made from compressed high purity exfoliated mineral graphite. The layer of anisotropic material 55 has an area that is larger (e.g., at least twice as large) as an area of the central section 10C. The layer of anisotropic material 55 is preferably both thermally conductive and electrically conductive, and it acts to spread both the flow of current and heat in all four directions (i.e., to the right, to the left, into the page, and out of the page in FIG. 2, which corresponds to right, left, up, and down in FIG. 1). The second layer of conductive adhesive 60 should be biocompatible, and its function is to hold the apparatus 100 against the subject's skin. Note that in alternative embodiments, a layer of conductive gel (e.g., hydrogel) can be used in place of the second layer of conductive adhesive 60 depicted in FIG. 2.

In alternative embodiments, the layer of anisotropic material 55 and the second layer of conductive adhesive 60 can be omitted, in which case the first layer of conductive adhesive 50 should be biocompatible so that it can be adhered directly to the subject's skin.

Positioning the temperature sensors T1-T8 at the peripheral section of the apparatus 100 is particularly advantageous in embodiments that include the layer of anisotropic material 55, because unlike the prior art embodiments (in which it is virtually certain that the hottest spot will be directly beneath an electrode element), embodiments that include a layer of anisotropic material 55 spread out the current and heat over a much larger surface. And as a result, additional factors, including but not limited to geometric factors and anatomical factors, may impact which portion of the apparatus 100 will be the hottest. In one example, geometry comes into play because portions of opposing transducer arrays that are closest to each other will tend to run hotter than the portions of opposing transducer arrays that are spaced further apart. In another example, anatomy comes into play because sections of the apparatus 100 that overlie portions of the subject's skin with lower blood flow will tend to run hotter than sections that overlie portions of the subject's skin with higher blood flow (because blood flow carries heat away from the apparatus 100). Optionally, one or more additional temperature sensors T9 can be positioned at the central section 10C of the apparatus 100.

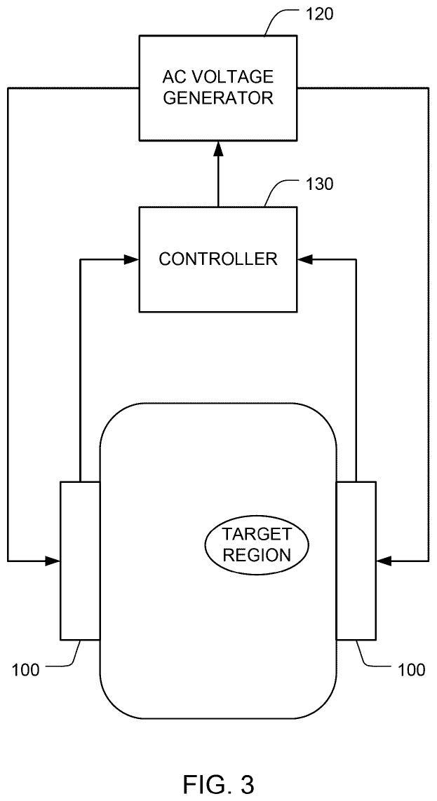

FIG. 3 depicts one example of how to use the apparatus 100 depicted in FIGS. 1-2 to apply alternating electric fields (e.g., TTFields) to a target region in a subject's body. Referring to FIGS. 1-3, a first apparatus 100 is adhered to the subject's skin on one side of the target region, and a second apparatus 100 is adhered to the subject's skin on the opposite side of the target region. To impose alternating electric fields in the target region, the AC voltage generator 120 applies an AC voltage (e.g., between 50 kHz and 5 MHz, or 75-300 kHz) between the metal pads 12 (FIGS. 1-2) of the first apparatus 100 and the metal pads 12 in the second apparatus 100. The AC signal from the AC voltage generator arrives at each of the first and second apparatuses 100 via a set of cables that terminate on the connector 15 of each apparatus 100. And from the connector 15, the signals are routed to the respective metal pads 12 via respective conductive traces (e.g., conductive metal traces, not shown) on the PCB 10.

The controller 130 ascertains the temperature of each of the temperature sensors T1-T8 (and T9, if present) in each of the first and second apparatuses 100 by inputting respective signals from those temperature sensors. Those signals arrive at the controller 130 via respective conductive traces on the respective PCB 10, respective connectors 15, and respective cables. If the controller 130 determines that all of the temperature sensors are below the threshold temperature, the controller 130 can increase the voltage of the AC signal, which will in turn increase the current that flows through the first and second apparatuses 100, which will in turn increase the intensity of the alternating electric field in the target region. On the other hand, if the controller 130 determines that any of the temperature sensors are at or approaching the threshold temperature, the controller 130 will decrease the voltage of the AC signal, which will decrease the current and eventually decrease the temperature of the hottest region of the apparatus 100.

Notably, the signals that the AC voltage generator 120 sends to the metal pads 12 of the first and second apparatuses 100 have relatively high currents (e.g., on the order of 1 A or even higher). On the other hand, the signals that the controller 130 uses to ascertain the temperature of each of the temperature sensors T1-T8 (and T9, if present) is orders of magnitude lower (e.g., on the order of 1 mA or even lower). As a result, the PCB 10 can be implemented using relatively thick metal traces (e.g., copper traces) to route the signals from the connector 15 to the metal pads 12, and relatively thin metal traces (e.g., copper traces) to route the signals from the connector 15 to the temperature sensors T1-T8 (and T9, if present). And while this is not problematic from a technical perspective, it is sub-optimal from an economic perspective.

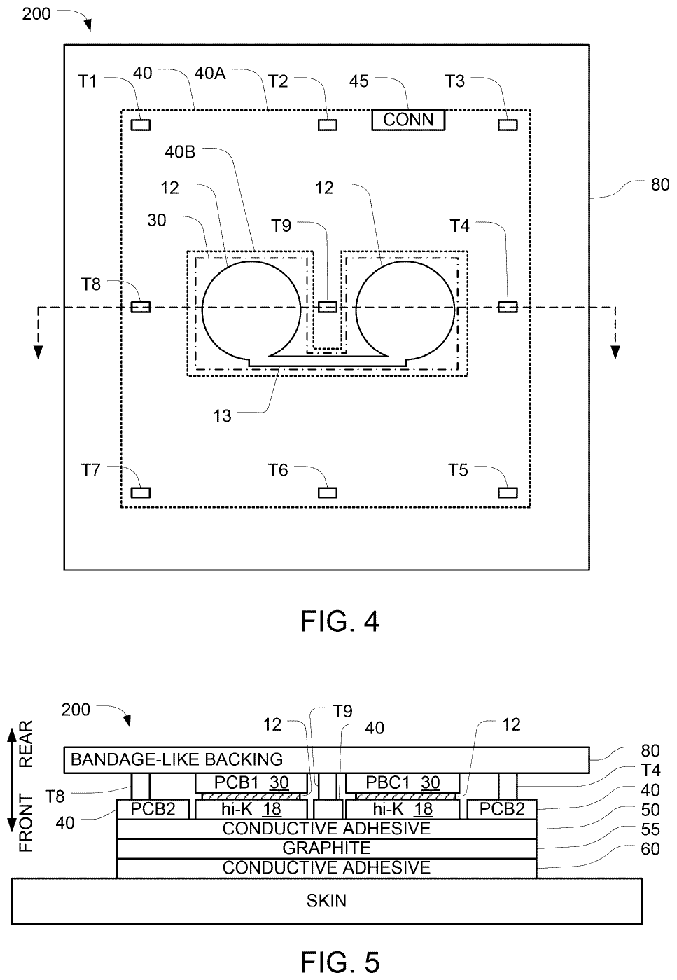

More specifically, because transducer arrays have relatively large areas (e.g., on the order of 10-50 square inches), when a single PCB 10 is used to both route signals to the metal pads 12 (for applying TTFields) and route signals from the temperature sensors T1-T8 (and T9, if present) (for obtaining temperature readings), the single PCB will be relatively expensive. This is because when a single PCB 10 is used, the entire PCB must be designed to accommodate the highest expected current, even though only a relatively small portion of the PCB is actually used to carry higher currents. And large PCBs that can accommodate currents on the order of 1 A are relatively expensive. The embodiments described below in connection with FIGS. 4-7 address this economic issue.

FIG. 4 is a plan view of another apparatus 200 (e.g., a transducer array) for applying alternating electric fields (e.g., TTFields) to a subject's body, and FIG. 5 is a section view of the same apparatus 200. The apparatus 200 includes a first PCB 30 positioned in the central portion of the apparatus, and a second PCB 40 positioned at the peripheral portion of the apparatus. The first PCB 30 has an outer boundary (shown as a -.- line), and the first and second PCBs are positioned so that the second PCB 40 lies outside the outer boundary of the first PCB 30.

The first PCB 30 in the central portion has a first substrate and at least one metal pad 12 disposed on the front face of the first substrate. When more than one metal pad 12 is included (as depicted in FIGS. 4-5), all the metal pads 12 can be connected by conductive traces (e.g., metal traces) 13. Conductive metal traces (e.g., copper traces, not shown) on the first PCB 30 are used to route signals arriving from an AC voltage generator to the metal pads 12. A connector (not shown) may be included on the first PCB 30 to input these signals, or the signals can be electrically connected directly to one of the metal pads 12 or metal traces 13 using a hard-wired connection.

The second PCB 40 in the peripheral portion of the apparatus 200 (having an outer boundary 40A and an inner boundary 40B, each showing as a dotted line in FIG. 4) is flexible, and it has a second substrate with a plurality of conductive traces disposed thereon. At least two temperature sensors T1-T8 (e.g., thermistors) are mounted to the second PCB 40, and these temperature sensors are electrically connected to at least one of the conductive traces. Optionally, one or more additional temperature sensors T9 (e.g., thermistors) may be positioned in the central portion of the apparatus 200 (e.g., on the second PCB 40, as depicted in FIG. 4). A connector 45 is mounted to the second PCB 40, and this connector 45 is used to provide an electrical interface with the thermistors T1-T9.

In the embodiment depicted in FIGS. 4-5, the first PCB 30 also includes a layer of dielectric material 18 disposed on and in front of the metal pads 12, and this layer of dielectric material 18 (labelled “hi-K” in FIG. 5) has a dielectric constant of at least 10. In some preferred embodiments, the layer of dielectric material 18 has a dielectric constant of at least 20 or at least 40.

A first layer of conductive adhesive 50 is disposed on and in front of both the first PCB 30 and the second PCB 40, and the rear surface of this conductive adhesive 50 adheres to the front surface of the dielectric material 18 (of the first PCB 30). In some embodiments, the layer of dielectric material 18 is not included (e.g., as described below in connection with FIG. 7). Note that in alternative embodiments, a layer of conductive gel (e.g., hydrogel) can be used in place of the first layer of conductive adhesive 50 depicted in FIG. 5.

A flexible backing 80 (e.g., a bandage-like backing) is positioned behind both the first PCB 30 and the second PCB 40, and this flexible backing 80 is configured to support both of those PCBs. At least a portion of the flexible backing 80 extends laterally beyond the first and second PCBs 30, 40 and the front of this portion is covered with a biocompatible adhesive that adheres to skin. This portion of the flexible backing 80 helps hold the apparatus 200 against the subject's skin. Although the flexible backing 80 and the boundaries of the first and second PCBs 30, 40 are generally depicted herein for simplicity to be square or rectangular, they could, of course, be any shape including circular, oval, rounded triangular, etc.

The embodiment depicted in FIGS. 4-5 also includes a layer of anisotropic material (e.g., a sheet of graphite) 55 that is disposed on and in front of the first layer of conductive adhesive 50, and a second layer of conductive adhesive 60 disposed on and in front of the layer of anisotropic material 55. The layer of anisotropic material 55 has an area that is larger (e.g., at least twice as large) as the area of the first PCB 30. The layer of anisotropic material 55 is preferably both thermally conductive and electrically conductive, and it acts to spread both the flow of current and heat in all four directions as described above in connection with FIGS. 1-2. The second layer of conductive adhesive 60 should be biocompatible, and its function is to hold the apparatus 200 against the subject's skin. Note that in alternative embodiments, a layer of conductive gel (e.g., hydrogel) can be used in place of the second layer of conductive adhesive 60 depicted in FIG. 5.

In alternative embodiments, the layer of anisotropic material 55 and the second layer of conductive adhesive 60 can be omitted, in which case the first layer of conductive adhesive 50 should be biocompatible so that it can be adhered directly to the subject's skin.

Positioning the temperature sensors T1-T8 at the peripheral section of the apparatus 200 is advantageous for the same reasons described above in connection with the FIGS. 1-2 embodiment.

Notably, unlike the FIGS. 1-2 embodiment in which low current PCB traces (which are used to interface with the temperature sensors T1-T9) are implemented on the same PCB as high current traces (which are used to interface with the metal pads 12), all of the traces on the second PCB 40 are low current traces. And this enables the second PCB 40 to be made using very inexpensive technologies (e.g., with conductive traces made from conductive ink instead of copper). Moreover, because all of the high current traces are located on the first PCB 30, which is relatively small, the FIGS. 4-5 embodiment can be implemented using a relatively small high-current PCB (using expensive copper technology) and a relatively large low-current PCB that uses a less expensive (e.g., conductive ink) technology. And because the size of the expensive-technology PCB 30 in the FIGS. 4-5 embodiment is much smaller than the single, large, expensive-technology PCB 10 in the FIGS. 1-2 embodiment, the former can deliver a significant cost savings with respect to the latter.

The apparatus 200 depicted in FIGS. 4-5 is used in a manner that is similar to how the apparatus 100 (depicted in FIGS. 1-2) is used, as described above in connection with FIG. 3, with one notable exception. More specifically, instead of using a single connector 15 to route signals to and from both the thermistors T1-T8 (and T9, if present) and the metal pads 12 (as in the FIGS. 1-2 embodiment), the FIGS. 4-5 embodiment uses the connector 45 on the second PCB 40 only to route signals to the thermistors T1-T8 (and, optionally, T9). And either an additional connector (not shown) or a hard-wired connection is used to route the AC TTFields signals to the metal pads 12.

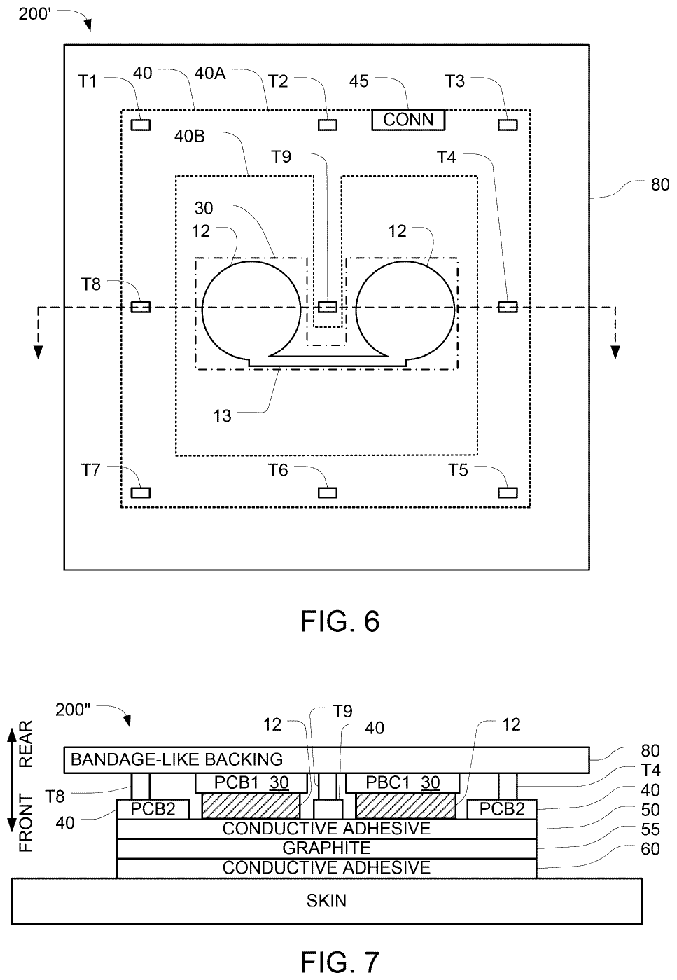

FIG. 6 depicts another apparatus 200′ that is similar to the apparatus 200 described above in connection with FIGS. 4-5, except that there is a larger space between the outer boundary of the first PCB 30 (shown as a -.- line) and the inner boundary 40B of the second PCB 40 (shown as a dotted line). Use of this apparatus 200′ (and the labelling of the components of 200′ in FIG. 6) is similar to the use of the apparatus 200 (and the labelling of the components of 200 in FIG. 4-5) described above.

FIG. 7 depicts yet another apparatus 200″ that is also similar to the apparatus 200 described above in connection with FIGS. 4-5, except that the layer of dielectric material 18 is omitted. As a result, the rear surface of the first layer of conductive adhesive 50 will adhere to the front surface of the metal pads 12 of the first PCB 30). This apparatus 200″ is used instead of the apparatus 200 described above in situations when it is desirable to conductively couple the AC signal from the AC voltage generator 120 (shown in FIG. 3) to the subject's body (as opposed to capacitively coupling that signal into the subject's body).

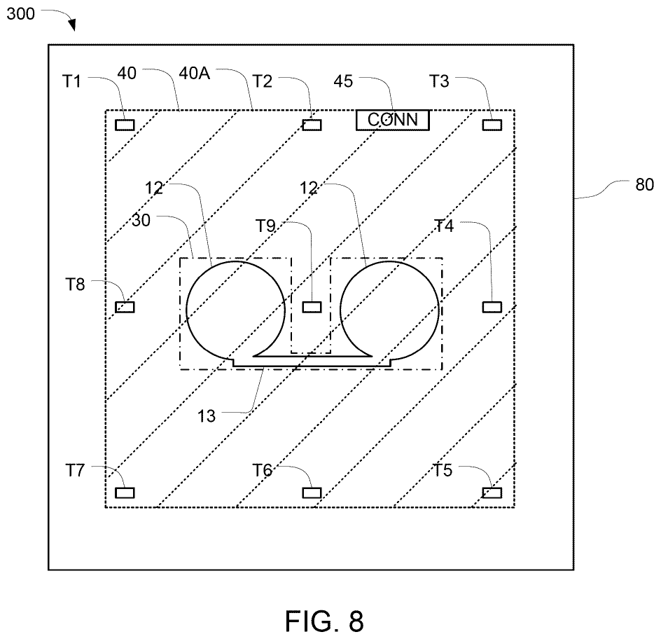

In the embodiments described above in connection with FIGS. 4 and 6, there is a space between the outer boundary of the first PCB 30 (shown as a -.- line) and the inner boundary 40B of the second PCB 40 (shown as a dotted line). This space is small in FIG. 4 and large in FIG. 6, and there is no portion of either FIG. 4 or 6 that shows the second PCB 40 overlapping the first PCB 30. But in alternative embodiments, the second PCB 40 can partially or completely overlap the first PCB 30. FIG. 8 is an example of such an embodiment 300. This FIG. 8 embodiment is similar to the FIG. 4 embodiment 200, except that the second PCB 40 (drawn with diagonal crosshatching) is rectangular with no cut-out area. Preferably, at places where the first and second PCBs 30, 40 overlap, the second PCB 40 is positioned behind the first PCB 30 (i.e., with the first PCB 30 in front and the second PCB 40 in the rear.) In this example, the first PCB 30 has an outer boundary (shown as a -.- line), the second PCB 40 has an outer boundary 40A (shown as a dotted line), and the first and second PCBs 30, 40 are positioned so that the outer boundary of the second PCB 40 lies outside the outer boundary of the first PCB 30 (when viewed from a direction perpendicular to the first PCB 30). In FIG. 8, the second PCB 40 completely overlaps the first PCB 30. Optionally, for embodiments where the second PCB 40 partially or completely overlaps the first PCB 30, the first and second PCBs 30, 40 may be laminated together. Optionally, the laminate may comprise a conductive adhesive between the first and second PCBs 30, 40.

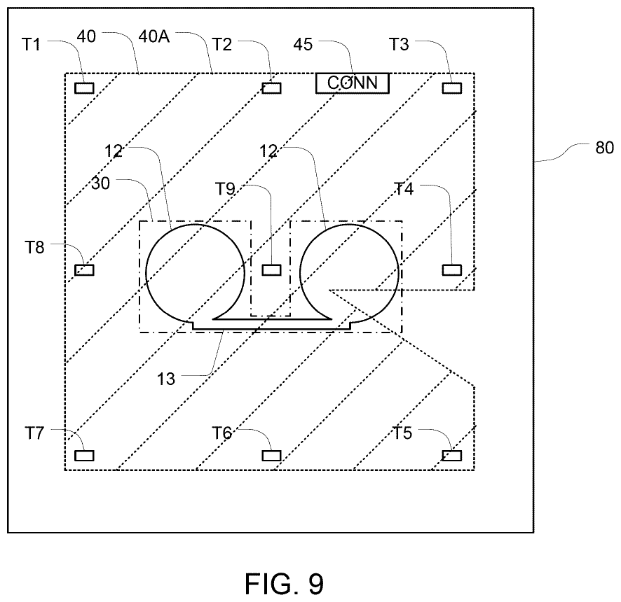

FIG. 9 is a variation of the FIG. 8 embodiment in which the second PCB 40 has an irregular shape. In this situation, the first PCB 30 has an outer boundary (shown as a -.- line), the second PCB 40 has an outer boundary 40A (shown as a dotted line), and the first and second PCBs 30, 40 are positioned so that a rectangle that most closely circumscribes the outer boundary of the second PCB 40 lies outside the outer boundary of the first PCB 30 (even though a portion of the first PCB 30 actually lies outside the outer boundary of the second PCB 40).

Headings are provided for convenience only and are not to be construed to limit the disclosure in any manner. Embodiments illustrated under any heading or in any portion of the disclosure may be combined with embodiments illustrated under the same or any other heading or other portion of the disclosure. Any combination of the elements described herein in all possible variations thereof is encompassed by the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus comprising:
a first PCB having a first substrate and at least one metal pad disposed on a skin-facing front face of the first substrate;
a second PCB having a second substrate and a plurality of conductive traces, wherein the second PCB is flexible; and
a plurality of temperature sensors, each of which is electrically connected to at least one of the conductive traces of the second PCB,
wherein the first PCB has an outer boundary, and wherein the first and second PCBs are positioned so that, when viewed from a direction perpendicular to the first PCB, the second PCB lies outside the outer boundary of the first PCB.

2. The apparatus of claim 1, further comprising a flexible backing positioned behind the first PCB and the second PCB, wherein the flexible backing is configured to support the first PCB and the second PCB, and wherein at least a portion of the flexible backing extends laterally beyond both the first PCB and the second PCB and is covered with a biocompatible adhesive that adheres to skin,
wherein the first PCB is flexible, and
wherein the plurality of conductive traces of the second PCB are formed using conductive ink.

3. The apparatus of claim 2, wherein the first PCB has a first area, the second PCB has a second area, and the second area is larger than the first area.

4. The apparatus of claim 2, further comprising:
a first layer of conductive adhesive or conductive gel disposed on and in front of both the first PCB and the second PCB;
a layer of anisotropic material disposed on and in front of the first layer of conductive adhesive or conductive gel; and
a second layer of conductive adhesive or conductive gel disposed on and in front of the layer of anisotropic material.

5. The apparatus of claim 4, wherein the layer of anisotropic material comprises graphite.

6. The apparatus of claim 4, wherein the layer of anisotropic material comprises a layer of pyrolytic graphite, graphitized polymer film, or graphite foil made from compressed high purity exfoliated mineral graphite.

7. The apparatus of claim 1, wherein each of the metal pads is a copper pad.

8. The apparatus of claim 1, further comprising at least one layer of dielectric material disposed in front of the at least one metal pad, optionally disposed on and in front of the at least one metal pad, wherein the at least one layer of dielectric material has a dielectric constant of at least 10.

9. The apparatus of claim 1, further comprising a connector affixed to the second PCB, wherein the plurality of temperature sensors, the plurality of conductive traces of the second PCB, and the connector are positioned and arranged so that temperature readings obtained using the plurality of temperature sensors can be accessed via the connector.

10. The apparatus of claim 1, wherein the first and second substrates partially or completely overlap one another in an overlap region and are laminated together in the overlap region.

11. The apparatus of claim 1, wherein the first and second substrates are the same substrate or wherein the first and second substrates are coplanar.

12. An apparatus for applying an electrical signal to a subject's body, the apparatus comprising:
a flexible PCB having a central section and a peripheral section located outside an outer boundary of the central section, wherein the PCB has at least one metal pad disposed on a skin-facing front face of the central section of the PCB, and a plurality of conductive traces;
at least two temperature sensors, each of which is mounted to the peripheral section of the PCB and electrically connected to at least one of the conductive traces;
a first layer of conductive adhesive or conductive gel disposed on and in front of the PCB; and
a flexible backing positioned behind the PCB, wherein the flexible backing is configured to support the PCB, and wherein at least a portion of the flexible backing extends laterally beyond the PCB and is covered with a biocompatible adhesive that adheres to skin.

13. The apparatus of claim 12, wherein the central section of the flexible PCB is bounded by a convex hull enclosing a perimeter of outermost metal pads of the at least one metal pad.

14. The apparatus of claim 12, further comprising:
a layer of anisotropic material disposed on and in front of the first layer of conductive adhesive or conductive gel, wherein the layer of anisotropic material has an area that is at least twice as large as an area of the central section; and
a second layer of conductive adhesive or conductive gel disposed on and in front of the layer of anisotropic material.

15. The apparatus of claim 14, wherein the layer of anisotropic material comprises graphite.

16. The apparatus of claim 12, wherein the PCB further comprises at least one layer of dielectric material disposed on the at least one metal pad and in front of the at least one metal pad, wherein the at least one layer of dielectric material has a dielectric constant of at least 10.

17. The apparatus of claim 12, wherein none of the at least one metal pad is located in the peripheral section of the PCB.

18. An apparatus for applying an electrical signal to a subject's body, the apparatus comprising:
a first PCB having a first substrate and at least one metal pad disposed on a skin-facing front face of the first substrate;
a second PCB having a second substrate and a plurality of conductive traces formed using conductive ink, wherein the second PCB is flexible;
a plurality of temperature sensors, each of which is electrically connected to at least one of the conductive traces of the second PCB; and
a flexible backing positioned behind the first PCB and the second PCB, wherein the flexible backing is configured to support the first PCB and the second PCB, and wherein at least a portion of the flexible backing extends laterally beyond both the first PCB and the second PCB and is covered with a biocompatible adhesive that adheres to skin,
wherein the first PCB has an outer boundary, the second PCB has an outer boundary, and wherein first and second PCBs are positioned so that a convex hull that most closely circumscribes the outer boundary of the second PCB lies outside the outer boundary of the first PCB.

19. The apparatus of claim 18, further comprising:
a first layer of conductive adhesive or conductive gel disposed on and in front of both the first PCB and the second PCB;
a layer of anisotropic material disposed on and in front of the first layer of conductive adhesive or conductive gel; and
a second layer of conductive adhesive or conductive gel disposed on and in front of the layer of anisotropic material.

20. The apparatus of claim 19, wherein the layer of anisotropic material comprises graphite.

21. The apparatus of claim 19, wherein the first PCB further comprises at least one layer of dielectric material disposed on the at least one metal pad and in front of the at least one metal pad, wherein the at least one layer of dielectric material has a dielectric constant of at least 10.
